(19) United States
(12) Patent Application Publication
(10) Pub. No.: US 2024/0398337 A1
(43) Pub. Date: Dec. 5, 2024

(54) STIMULUS ADJUSTMENT APPARATUS, STIMULUS ADJUSTMENT METHOD, AND STIMULUS ADJUSTMENT PROGRAM

(71) Applicant: NIPPON TELEGRAPH AND TELEPHONE CORPORATION, Tokyo (JP)

(72) Inventor: Arinobu NIIJIMA, Musashino-shi, Tokyo (JP)

(73) Assignee: NIPPON TELEGRAPH AND TELEPHONE CORPORATION, Tokyo (JP)

(21) Appl. No.: 18/690,755

(22) PCT Filed: Oct. 6, 2021
(86) PCT No.: PCT/JP2021/037028

§ 371 Date: Mar. 11, 2024

(51) Int. Cl. A61B 5/00; A61B 5/0531; G16H 20/30

(52) U.S. Cl. CPC A61B 5/4836; A61B 5/0531; G16H 20/30

(57) ABSTRACT

Provided are a stimulation adjustment device, a stimulation adjustment method, and a stimulation adjustment program that can automatically adjust the intensity of stimulation presented to a user. A stimulation adjustment device according to one embodiment includes: a signal input unit to which a measurement signal of electrodermal activity of a user is input; an analysis unit that analyzes the electrodermal activity on the basis of the measurement signal; an adjustment unit that generates an instruction signal for adjusting an intensity of stimulation presented to the user according to a result of the analysis of the electrodermal activity; and a signal output unit that outputs the instruction signal.

### TECHNICAL FIELD

Embodiments of the present invention relate to a stimulation adjustment device, a stimulation adjustment method, and a stimulation adjustment program.

### BACKGROUND ART

Systems for presenting tactile stimulation to humans for various purposes such as treatment, training, beauty, and entertainment are known. Such systems require pre-calibration such that the intensity of the tactile stimulation is appropriate for the purpose.

For example, when electrical muscle stimulation (EMS) techniques are used and electrical stimulation is presented to muscles to cause involuntary contraction in the muscles, the stimulation intensity should be adjusted to a range in which a user does not feel pain. A technique for pre-calibrating the stimulation intensity in advance in a system that uses EMS to control a user's hand movements for the purpose of assisting with learning to play a musical instrument has been proposed (see, for example, NPL 1).

### CITATION LIST

### Non Patent Literature

[NPL 1] Emi Tamaki, Takashi Miyaki, and Jun Rekimoto, “Possessed Hand: Techniques for Controlling Human Hands using Electrical Muscles stimuli”, Proceedings of the SIGCHI Conference on Human Factors in Computing Systems. 2011
[NPL 2] Ebrahim Babaei et al. “A Critique of Electrodermal Activity Practices at CHI”, Proceedings of the 2021 CHI Conference on Human Factors in Computing Systems. May 8-13, 2021.

### SUMMARY OF INVENTION

### Technical Problem

In the related art, the intensity of tactile stimulation is usually adjusted by a direct operation by the user himself or herself. However, in the adjustment by the user's self-reporting, there is a risk that the user will mistakenly increase the stimulation intensity even though he or she is actually feeling pain, and the system will be used while causing pain.

Further, even when the user does not feel pain during the pre-calibration, long-term use may cause pain as the skin sweats and becomes more conductive to electricity, and pain may occur due to accumulated muscle fatigue. In such a case, if calibration by the user himself or herself is required again, it is inconvenient.

The present invention has been made in view of the above circumstances, and an object of the present invention is to provide a stimulation adjustment device, a stimulation adjustment method, and a stimulation adjustment program that can automatically adjust the intensity of stimulation presented to a user.

### Solution to Problem

According to one embodiment of the present invention, there is provided a stimulation adjustment device including: a signal input unit to which a measurement signal of electrodermal activity of a user is input; an analysis unit configured to analyze the electrodermal activity on the basis of the measurement signal; an adjustment unit configured to generate an instruction signal for adjusting an intensity of stimulation presented to the user according to a result of the analysis of the electrodermal activity; and a signal output unit configured to output the instruction signal.

### Advantageous Effects of Invention

A stimulation adjustment device according to one embodiment outputs an instruction signal for adjusting the intensity of stimulation presented to a user on the basis of an analysis result of electrodermal activity of the user. It is known that the value of a user's electrodermal activity (EDA) changes when psychogenic perspiration occurs, such as when the user feels pain (see NPL 2). Therefore, by analyzing the user's electrodermal activity, it is possible to estimate the degree of psychogenic perspiration and the degree of pain of the user. Therefore, the stimulation adjustment device according to one embodiment of the present invention can output an instruction signal for automatically adjusting the intensity of the stimulation in consideration of the degree of pain of the user without requiring a direct operation from the user.

That is, according to the present invention, it is possible to provide a stimulation adjustment device, a stimulation adjustment method, and a stimulation adjustment program that can automatically adjust the intensity of stimulation presented to a user.

### BRIEF DESCRIPTION OF DRAWINGS

FIG. 1 is a block diagram illustrating an example of an overall configuration of a system provided with a stimulation adjustment device according to one embodiment.

FIG. 2 is a block diagram illustrating an example of a hardware configuration of the stimulation adjustment device according to one embodiment.

FIG. 3 is a flowchart illustrating an example of an information processing operation by the stimulation adjustment device according to one embodiment.

FIG. 4 is a diagram illustrating a result of verification of a relationship between stimulation intensity and electrodermal activity.

### DESCRIPTION OF EMBODIMENTS

Hereinafter, an embodiment according to the present invention will be described with reference to the drawings. Elements that are the same as or similar to elements that have already been described are denoted by the same or similar reference signs, and repeated description will be basically omitted.

### Embodiment

### (1) Configuration

### (1-1) System

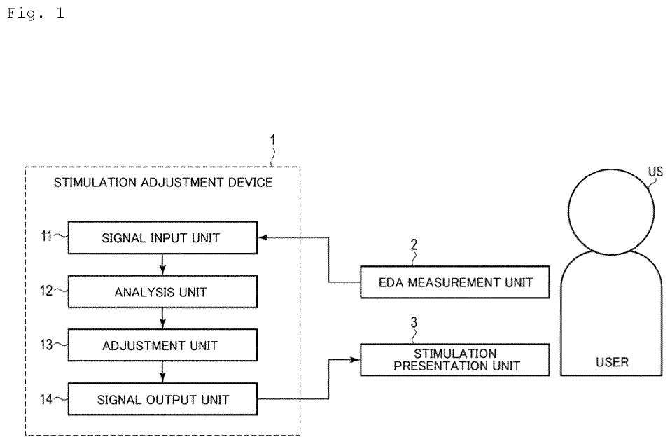

FIG. 1 is a block diagram illustrating an example of an overall configuration of a system provided with a stimulation adjustment device 1 according to one embodiment. This system includes a stimulation adjustment device 1, an EDA measurement unit 2, and a stimulation presentation unit 3. The EDA measurement unit 2 measures electrodermal activity (EDA) of a user US and outputs a measurement signal. The EDA measurement unit 2 is, for example, a device including an EDA sensor having an electrode to be stuck on a skin surface of the user US. The EDA sensor may measure various types of electrodermal activity. For example, the EDA sensor includes a pair of electrodes and measures a potential difference between different points of the skin. The EDA measurement unit 2 performs predetermined processing including amplification, noise removal, AD conversion, and the like on a signal obtained from the EDA sensor, and outputs a measurement signal of the electrodermal activity. The EDA measurement unit 2 is connected to a signal input terminal (not illustrated) of the stimulation adjustment device 1 via a signal cable, for example. The EDA measurement unit 2 may transmit a measurement signal to the stimulation adjustment device 1 by short-range radio communication or the like.

The stimulation presentation unit 3 presents tactile stimulation to the user US. The stimulation presentation unit 3 may present any of a wide variety of tactile stimuli. The stimulation presentation unit 3 is, for example, a presentation device of electrical muscle stimulation (EMS) including an electrode to be stuck on the skin surface of the user US. The presentation device of the EMS can be, for example, a device that presents the EMS to the user US for a wide variety of purposes, such as an electrical stimulation therapy device, an auxiliary device for sports training, or a learning auxiliary device for musical instrument performance. The stimulation presentation unit 3 presents stimulation to the user US according to an instruction signal output from the stimulation adjustment device 1. The instruction signal includes instructions (commands) related to adjustment of the intensity of stimulation. The instruction signal may also include other information about the stimulation, such as a presentation pattern of the stimulation. The stimulation presentation unit 3 is connected to a signal output terminal (not illustrated) of the stimulation adjustment device 1 via, for example, an arbitrary signal cable, and receives an instruction signal from the stimulation adjustment device 1. The stimulation presentation unit 3 may receive an instruction signal from the stimulation adjustment device 1 by short-range radio communication or the like. The stimulation presentation unit 3 may be configured to be able to present stimulation according to an instruction received through a manual operation or the like of a user in addition to an instruction signal received from the stimulation adjustment device 1.

The stimulation adjustment device 1 can be implemented as a microcomputer including a central processing unit (CPU) and a memory, for example. According to one embodiment, the stimulation adjustment device 1 includes a signal input unit 11, an analysis unit 12, an adjustment unit 13, and a signal output unit 14.

The signal input unit 11 receives a measurement signal of the electrodermal activity of the user US from the EDA measurement unit 2 and passes the measurement signal to the analysis unit 12.

The analysis unit 12 analyzes the electrodermal activity of the user US on the basis of the measurement signal input to the signal input unit 11. In one embodiment, the process of analyzing the electrodermal activity includes a process of comparing a value of the electrodermal activity obtained from the measurement signal with a preset threshold value and determining whether or not the value of the electrodermal activity exceeds the threshold value. The value of the electrodermal activity reflects the degree of psychogenic perspiration of the user US, and the degree of psychogenic perspiration is considered to be greater as the degree of pain felt by the user US is stronger. The threshold value may be set arbitrarily according to the purpose of stimulation presentation. For example, the threshold value is set based on a value of electrodermal activity measured in a state where no stimulation is presented to the user US. As an example, a value a certain percentage or a certain value higher than a reference value of electrodermal activity measured in a state where no stimulation is presented to the user US may be set as the threshold value. The analysis unit 12 passes the analysis result to the adjustment unit 13. The analysis result can be referred to as a comparison result of whether or not the value of the electrodermal activity exceeds a threshold value. The analysis result may include other information. The analysis result may be replaced with other information reflecting the degree of pain of the user US.

The adjustment unit 13 generates an instruction signal for adjusting the intensity of stimulation presented to the user US according to the analysis result of the electrodermal activity received from the analysis unit 12. The instruction signal includes, for example, information indicating the intensity of the stimulation after adjustment. The intensity of stimulation can be adjusted by controlling the current, voltage, frequency, pulse width, or the like associated with the stimulation. In one embodiment, upon receiving an analysis result indicating that the value of the electrodermal activity exceeds a predetermined threshold value from the analysis unit 12, the adjustment unit 13 determines that adjustment to weaken the intensity of stimulation presented to the user US is necessary, and generates an instruction signal for instructing the adjustment to weaken the intensity of the stimulation.

The signal output unit 14 outputs the instruction signal generated by the adjustment unit 13. The instruction signal output by the signal output unit 14 is received by the stimulation presentation unit 3, and the stimulation presentation unit 3 adjusts the stimulation presented to the user US according to the instruction signal.

Note that the system configuration illustrated in FIG. 1 is merely an example. The stimulation adjustment device 1 may include a part of the EDA measurement unit 2 or a part of the stimulation presentation unit 3. The stimulation adjustment device 1 may be integrated with the EDA measurement unit 2 or the stimulation presentation unit 3.

### (1-2) Hardware Configuration of Stimulation Adjustment Device

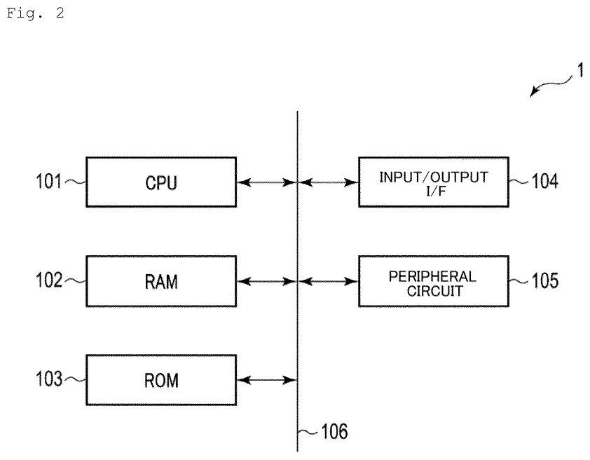

FIG. 2 is a block diagram illustrating an example of a hardware configuration of the stimulation adjustment device 1. In the illustrated example, the stimulation adjustment device 1 includes a CPU 101, a random access memory (RAM) 102, a read only memory (ROM) 103, an input/output interface (input/output I/F) 104, and a peripheral circuit 105. The CPU 101, the RAM 102, the ROM 103, the input/output interface (input/output I/F) 104, and the peripheral circuit 105 are electrically connected by a bus 106.

The CPU 101 controls the overall operation of the stimulation adjustment device 1. The RAM 102 is, for example, a volatile semiconductor memory and is used as a work area of the CPU 101. The ROM 103 is a non-volatile semiconductor memory, and holds a program, control data, and the like for controlling the stimulation adjustment device 1. The CPU 101 loads the program stored in the ROM 103 in the RAM 102, and interprets and executes the loaded program, thereby implementing the signal input unit 11, the analysis unit 12, the adjustment unit 13, and the signal output unit 14 described above.

The input/output interface 104 is an interface for connecting the stimulation adjustment device 1 and the EDA measurement unit 2, and connecting the stimulation adjustment device 1 and the stimulation presentation unit 3. The input/output interface 104 includes input/output terminals for analog or digital signals. The input/output interface 104 may include an interface for wired or wireless communication.

The peripheral circuit 105 includes a timer, a counter, and the like. The peripheral circuit 105 may also include an A/D converter.

Regarding a specific hardware configuration of the stimulation adjustment device 1, it is possible to appropriately omit, replace, and add components according to the embodiment. For example, there may be two or more CPUs 101 instead of one. Also, instead of the CPU 101, an application specific integrated circuit (ASIC), a field-programmable gate array (FPGA), or the like may be used.

### (2) Operation

Next, an example of an information processing operation of the stimulation adjustment device 1 according to one embodiment will be described.

As a premise of the operation, the stimulation presentation unit 3 is associated with the body of the user US. The stimulation presentation unit 3 may be associated with any location of the body of the user US. The following description is made on the assumption that the stimulation presented via the stimulation presentation unit 3 is tactile stimulation, particularly electrical muscle stimulation (EMS). For example, electrodes of the stimulation presentation unit 3 that presents EMS are attached to the forearm of the user US.

Moreover, as a premise of the operation, the EDA measurement unit 2 is also associated with the body of the user US. The part of the body where EDA is measured may be any part where psychogenic perspiration can be measured. Parts where psychogenic perspiration can be measured include, but are not limited to, finger pads, palms, and soles. For example, the EDA measurement unit 2 receives potential signals from a pair of electrodes attached to the fingers of the user US, acquires an analog signal representing the potential difference between two points, performs predetermined processing such as amplification, noise removal, and AD conversion, and transmits the EDA measurement signal to the stimulation adjustment device 1.

Note that the method of measuring the potential difference between two electrodes without current flow as in the above example is called a potential method. One example of EDA measured by the potential method is skin potential reflex (SPR), which is known as a transient skin potential response. SPR is known to have a linear relationship with the intensity of stimulation related to pain sensation and the like. However, the present invention is not limited thereto, and the stimulation adjustment device 1 according to one embodiment can be applied to various EDA measurements. For example, another example of EDA measured by the potential method is a skin potential level (SPL), which is said to reflect arousal level. EDA may also be measured by an electrification method in which a weak current is applied and the resistance or conductance response of the skin is measured. Examples of EDA measured by the electrification method are a skin resistance level (SRL), a skin resistance response (SRP), a skin conductance level (SCL), and a skin conductance response (SCR).

Furthermore, here, it is assumed that a threshold value for the user US is set in advance by activating only the EDA measurement unit 2 first, measuring the EDA in a state where no stimulation is presented to the user US, and using its measurement value as a reference value. For example, the threshold value is set as a value higher than the reference value by an arbitrary percentage (X%) (for example, 20% from the reference value). However, this is only an example, and the threshold value may be set by other methods. Also, the threshold value may be set for each user, or may be set commonly among a plurality of users. The threshold value may be calculated and set by a function unit (not illustrated) of the stimulation adjustment device 1, or may be set by manual input of an operator.

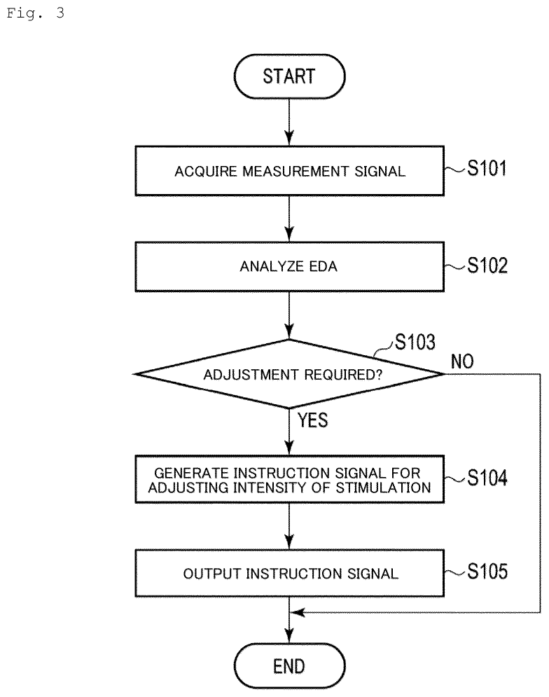

FIG. 3 is a flowchart illustrating an example of an information processing operation by the stimulation adjustment device 1 according to one embodiment. The subsequent processing is executed in a state where the stimulation presentation unit 3 is also activated in addition to the EDA measurement unit 2 and the stimulation is presented to the user US.

First, in step S101, the stimulation adjustment device 1 acquires a measurement signal output from the EDA measurement unit 2 via the signal input unit 11, for example, for a certain period of time.

In step S102, the analysis unit 12 of the stimulation adjustment device 1 analyzes the EDA of the user US on the basis of the acquired measurement signal, and passes the analysis result to the adjustment unit 13. In one embodiment, the analysis unit 12 of the stimulation adjustment device 1 compares an EDA value obtained from the measurement signal with a threshold value, and passes the comparison result to the adjustment unit 13 as an analysis result. In a more specific example, the analysis unit 12 calculates a moving average of the EDA value using an analysis window having a predetermined width on the basis of the input measurement signal, and compares the calculated moving average with a preset threshold value. When EDA is measured by using the electrodes as described above, spike noise may occur in the measurement data if the electrodes are displaced due to body movement. By calculating the moving average, the influence of noise caused by such body movement can be suppressed.

However, the use of the moving average value is only an example, and other information obtained from the measurement signal may be used. For example, the stimulation adjustment device 1 may compare the maximum value, minimum value, variation width (difference between the maximum value and the minimum value), or the like of the measurement signal obtained for a predetermined analysis window with a threshold value.

In step S103, the adjustment unit 13 of the stimulation adjustment device 1 determines whether or not adjustment of the stimulation intensity is necessary on the basis of the analysis result received from the analysis unit 12. When it is determined that the adjustment of the stimulation intensity is necessary (YES), the process proceeds to step S104. When it is determined that the adjustment is not necessary (NO), the process ends.

Here, in one embodiment, the stimulation adjustment device 1 estimates that the user US is likely to feel pain when the moving average value of the EDA exceeds a threshold value, and therefore determines that the adjustment of stimulation intensity is necessary. The stimulation adjustment device 1 may immediately determine that the adjustment of the stimulation intensity is necessary when even one moving average value exceeds the threshold value, or determine that the adjustment of the stimulation intensity is necessary when the moving average value exceeds the threshold value continuously for a predetermined number or for a predetermined period.

In step S104, the adjustment unit 13 of the stimulation adjustment device 1 generates an instruction signal for adjusting the intensity of the stimulation, and passes the instruction signal to the signal output unit 14. In one embodiment, the instruction signal includes information indicating the intensity of the stimulation after adjustment. For example, the instruction signal may include information instructing the adjustment rate for current, voltage, frequency, pulse width, or the like, associated with stimulation. The information instructing the adjustment rate is, for example, information instructing to reduce the value by Y%. The instruction signal may include information instructing a value to be reduced, a level to be reduced, a value after adjustment, a level after adjustment, or the like for current, voltage, frequency, pulse width, or the like associated with stimulation.

In one embodiment, the system including the stimulation adjustment device 1 performs adjustment to weaken the intensity of EMS when the moving average value of EDA measurement values exceeds a threshold value. For example, the intensity of EMS can be weakened by lowering the current, lowering the voltage, lowering the frequency, or reducing the pulse width. As an example, an adjustment method of uniformly reducing the EMS intensity by a predetermined percentage (for example, 10%) (for example, weakening the current from 10 mA to 9 mA) is conceivable when the moving average value of the EDA measurement values exceeds the threshold value.

Alternatively, the system including the stimulation adjustment device 1 may perform adjustments depending on the ratio of the excess to the threshold value when the moving average value of the EDA values exceeds the threshold value. For example, adjustments may be performed such as reducing the intensity by 10% when the moving average value exceeds 110% of the threshold value, and reducing the intensity by 20% when the moving average value exceeds 120% of the threshold value. Alternatively, a plurality of threshold values may be set, and adjustment may be performed such that the intensity is reduced by As when the value of the EDA exceeds a first threshold value, and the intensity is reduced by B% when the value of EDA exceeds a second threshold value. Thus, adjustment can be performed in consideration of the degree of excess to the threshold value. What kind of adjustment should be performed according to what kind of analysis result may be arbitrarily set according to the purpose of presenting the stimulation or the like.

In step S105, the signal output unit 14 of the stimulation adjustment device 1 outputs the generated instruction signal. The instruction signal is received, for example, by a control unit (not illustrated) of the stimulation presentation unit 3. The stimulation presentation unit 3 adjusts the intensity of the electrical stimulation presented to the user US according to the instruction signal.

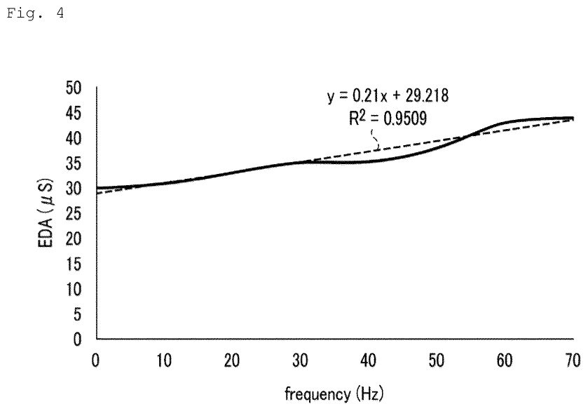

FIG. 4 illustrates a result of verification of a relationship between stimulation intensity and electrodermal activity (EDA). In FIG. 4, the horizontal axis represents frequency [Hz], and the vertical axis represents the value [μS] of EDA. In this example, the electrodes of the EDA sensor were attached to the index finger and the middle finger of the left hand of one subject, and the EMS was presented to the right forearm. For presentation of stimulation by EMS, a method (pulse frequency modulation: PFM) was employed in which a current value of 12 mA and a pulse width of 200 μs were fixed, and the stimulation intensity was adjusted by changing the frequency. The solid line shows the change in the EDA measurement values when the frequency is increased to 70 Hz every 10 Hz. It was observed that the higher the frequency associated with the stimulation of EMS, the greater the value of EDA. This is estimated to be because the higher the frequency, the higher the stimulation intensity of EMS, the stronger the pain felt by the subject, the more psychogenic perspiration increased, and the higher the EDA value. The broken line is an approximate straight line (y=0.21x+29.218, R²=0.9509) of the verification result.

Since the substantially linear correlation is observed between the stimulation intensity and the EDA measurement value, it is possible to easily predict how much the EDA measurement value is lowered by the adjustment amount of the stimulation intensity.

In the application of the stimulation adjustment device 1 according to the embodiment, there is no particular restriction in the relationship between the part where the EDA is measured and the part where the EMS is presented. For example, the EDA measurement unit 2 and the stimulation presentation unit 3 may be attached to a hand on the same side of the user US, or one of them may be attached to a hand and the other may be attached to separate parts such as a foot.

In addition, in the application of the stimulation adjustment device 1 according to the embodiment, the tactile stimulation presented to the user US is not limited to electrical stimulation. Since the tactile stimulation may be painful, in addition to the electrical stimulation, temperature stimulation (temperature that can stimulate pain sensation), pressure stimulation accompanied by tightening, and the like may be used. When presenting temperature stimulation, the stimulation presentation unit 3 may be a thermotherapy device, a cooling therapy device, or the like. When the Peltier element is used, warm or cold stimulation can be easily presented by current or voltage control, and can be easily adjusted using instruction signals. When presenting pressure stimulation, the stimulation presentation unit 3 may be a massager, a pressure wave therapy device, or the like. Even when presenting the pressure stimulation, the stimulation intensity can be easily adjusted by adjusting the voltage, current, frequency, pulse width, or the like of the control signal.

### (3) Effect

As described above in detail, in one embodiment of the present invention, a sensor capable of measuring electrodermal activity is attached to the user, the electrodermal activity of the user is analyzed on the basis of the measurement signal, and the intensity of the stimulation presented to the user is adjusted according to the result. This is based on the fact that when the user feels pain, psychogenic perspiration occurs according to the intensity of the pain, and when the psychogenic perspiration occurs, the value of the electrodermal activity changes. This makes it possible to automatically weaken the intensity of stimulation in consideration of psychogenic perspiration, which is an involuntary reaction, without requiring user's self-reporting or readjustment by the user himself or herself.

There is an individual difference in the way of feeling pain, and the way of feeling pain may change with time. According to one embodiment, a threshold value suitable for each user can be used by measuring electrodermal activity in a state where no stimulation is previously presented to the user and setting the threshold value with the measurement value as a reference value. Further, by setting a threshold value in advance, pain changing with time can be considered.

Also, depending on the purpose of stimulation presentation, there are cases where pain should be reduced as much as possible, and there are cases where some pain is tolerated. By appropriately setting the threshold value according to the purpose of the stimulation presentation, the stimulation presentation can be performed while achieving the purpose while reducing the burden on the user.

### Other Embodiments

The present invention is not limited to the above-described embodiment.

For example, the embodiments are applicable not only to tactile stimulation but also to other various stimulation presentations. It is known that psychogenic perspiration that can be evaluated by EDA is related not only to pain but also to stress, tension, anxiety, and the like. Thus, the above embodiments may be used in any field in which the stimulation can be adjusted in consideration of psychogenic perspiration. For example, the stimulation adjustment device 1 according to the embodiments may be used to adjust the intensity of visual stimulation, auditory stimulation, olfactory stimulation, or gustatory stimulation. As an example, the stimulation adjustment device 1 may be configured to monitor EDA measurement values of the user US while presenting visual stimulation to the user US for purposes such as diagnosis, testing, or experimentation, estimate that the user's stress is high when the EDA value exceeds the threshold value, and perform adjustment to reduce brightness, saturation, and the like.

It should be noted that the functions of the stimulation adjustment device 1 may be distributed to a plurality of devices, and these devices may cooperate with each other to perform processing. Further, each functional unit may be implemented by using a circuit. The circuit may be a dedicated circuit that implements a specific function, or may be a general-purpose circuit such as a processor.

Furthermore, the flow of processing described above is not limited to the described procedure, and the order of some steps may be changed, and some steps may be performed in parallel. Further, the series of processing described above need not be executed continuously in terms of time, each step may be executed at any timing.

The method described above can be stored in a recording medium (storage medium) such as a magnetic disk (Floppy (registered trademark) disk, hard disk, and the like), an optical disc (CD-ROM, DVD, MO, and the like), or a semiconductor memory (ROM, RAM, flash memory, and the like) as a program (software means) that can be executed by a computing machine (computer), and can also be distributed by being transmitted through a communication medium. Note that the programs stored on the medium side also include a setting program for configuring, in the computing machine, a software means (including not only an execution program but also tables and data structures) to be executed by the computing machine. The computing machine that implements the above device executes the above-described processing by reading the programs recorded in the recording medium, constructing the software means by the setting program as needed, and controlling the operation by the software means. Note that the recording medium described in the present specification is not limited to a recording medium for distribution, but includes a storage medium such as a magnetic disk or a semiconductor memory provided in the computing machine or in a device connected via a network.

Note that the present invention is not limited to the above embodiment, and can be modified in various ways without departing from the scope thereof at the implementation stage. In addition, the embodiments may be combined as appropriate, and in such a case, combined effects can be achieved. In addition, the embodiments described above include various aspects of the invention, and the various aspects of the invention can be extracted by combinations selected from a plurality of disclosed constituent elements. For example, even when some of all the constituent elements disclosed in the embodiments are deleted, as long as the problems can be solved and the effects can be obtained, a configuration from which the constituent elements are deleted can be extracted as an aspect of the invention.

### REFERENCE SIGNS LIST

1 Stimulation adjustment device
2 Electrodermal activity (EDA) measurement unit
3 Stimulation presentation unit
11 Signal input unit
12 Analysis unit
13 Adjustment unit
14 Signal output unit
101 CPU
102 RAM
103 ROM
104 Input/output interface
105 Peripheral circuit
106 Bus

1. A stimulation adjustment device, comprising:
signal input circuitry to which a measurement signal of electrodermal activity of a user is input;
analysis circuitry configured to analyze the electrodermal activity on the basis of the measurement signal;
adjustment circuitry configured to generate an instruction signal for adjusting an intensity of stimulation presented to the user according to a result of the analysis of the electrodermal activity; and
signal output circuitry configured to output the instruction signal.

2. The stimulation adjustment device according to claim 1, wherein:
the analysis circuitry analyzes the electrodermal activity by comparing a value of the electrodermal activity obtained from the measurement signal with a threshold value and determining whether or not the value of the electrodermal activity exceeds the threshold value, and
the adjustment circuitry generates an instruction signal instructing to weaken the intensity of the stimulation when it is determined that the value of the electrodermal activity exceeds the threshold value.

3. The stimulation adjustment device according to claim 2, wherein:
the threshold value is set based on the value of the electrodermal activity measured in a state where no stimulation is presented to the user.

4. The stimulation adjustment device according to claim 1, wherein;
the stimulation presented to the user is tactile stimulation, and
the measurement signal includes information of the electrodermal activity of the user measured in a state where the tactile stimulation is presented to the user.

5. The stimulation adjustment device according to claim 4, wherein:
the tactile stimulation includes electrical muscle stimulation, temperature stimulation, or pressure stimulation.

6. The stimulation adjustment device according to claim 1, wherein;
the adjustment circuitry generates an instruction signal instructing to reduce a value of current, voltage, frequency, or pulse width associated with the stimulation presented to the user.

7. A stimulation adjustment method executed by a computer, the method comprising:
acquiring a measurement signal of electrodermal activity of a user;
analyzing the electrodermal activity on the basis of the measurement signal;
generating an instruction signal for adjusting an intensity of stimulation presented to the user according to a result of the analysis of the electrodermal activity; and
outputting the instruction signal.

8. A non-transitory computer readable medium storing a stimulation adjustment program for causing a computer to perform the method of claim 7.
